(19) United States
(12) Patent Application Publication
(10) Pub. No.: US 2020/0377881 A1
(43) Pub. Date: Dec. 3, 2020

(54) Methods of Genome Engineering by Nuclease-Transposase Fusion Proteins

(71) Applicant: President and Fellows of Harvard College, Cambridge, MA (US)

(72) Inventors: Ellen L. Shrock, Brookline, MA (US); George M. Church, Brookline, MA (US); Eriona Hysolli, East Haven, CT (US)

(21) Appl. No.: 16/497,130

(22) PCT Filed: Mar. 23, 2018
(86) PCT No.: PCT/US18/24001

§ 371 Date: Sep. 24, 2019

Related U.S. Application Data
(60) Provisional application No. 62/475,989, filed on Mar. 24, 2017.

(51) Int. Cl. C12N 15/113; C12N 9/22; C12N 13/00; C12N 5/077

(52) U.S. Cl. CPC C12N 15/113; C12N 9/22; C12N 2800/80; C12N 5/0656; C12N 2310/20; C12N 13/00

(57) ABSTRACT

The present disclosure provides methods and compositions of altering a target nucleic acid sequence in a cell. The methods comprise introducing into the cell a guide RNA comprising a portion that is complementary to all or a portion of the target nucleic acid sequence, introducing into the cell a Cas9 transposase fusion protein, and introducing into the cell a donor nucleic acid sequence, wherein the guide RNA and the Cas9 transposase fusion protein co-localize at the target nucleic acid sequence, wherein the Cas9 transposase fusion protein cleaves the target nucleic acid sequence and the donor nucleic acid sequence is inserted into the target nucleic acid sequence in a site specific manner.

### RELATED APPLICATION DATA

This application claims priority to U.S. Provisional Application No. 62/475,989 filed on Mar. 24, 2017, which is hereby incorporated herein by reference in its entirety for all purposes.

### STATEMENT OF GOVERNMENT INTERESTS

This invention was made with government support under 5RM1HG008525-02 awarded by National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 22, 2018, is named 010498_01063_WO_SL.txt and is 6,507 bytes in size.

### FIELD

The present invention relates in general to methods of genome engineering by nuclease-transposase fusion proteins.

### BACKGROUND

Integration of genetic elements into the genome of a cell or a target DNA can be accomplished by a variety of methods. Some methods result in efficient integration at random genomic sites, while other methods result in integration at specific genomic loci. The latter methods are largely inefficient which involve constraints on the size of the payload and/or require multiple rounds of genetic modification.

Genome engineering of a cell mediated by sequence-specific nucleases is known. A nuclease-mediated double-stranded DNA (dsDNA) break in the genome can be repaired by two main mechanisms: non-homologous end joining (NHEJ) and homology directed repair (HDR).

Alternative methods have been developed to accelerate the process of genome engineering by directly injecting DNA or mRNA encoding site-specific nucleases into a cell such as a one cell embryo to generate DNA double strand break (DSB) at a specified locus in various species. DSBs induced by these site-specific nucleases can then be repaired by either non-homologous end joining (NHEJ) or homology directed repair (HDR). If a donor plasmid with homology to the ends flanking the DSB is co-injected, high-fidelity homologous recombination can produce animals with targeted integrations. A number of nucleases including zinc finger nucleases (ZNFs), transcription activator-like effector nucleases (TALENs) or CRISPR Cas nucleases are known to generate double stranded breaks in the genome and alter the target nucleic acid sequences in a site-specific manner. However, there is a continuing need for methods for efficient, targeted integration of multi-kilobase (and larger) genetic elements for routine and large-scale genome engineering in cells.

### SUMMARY

Aspects of the present disclosure relate to a method of altering a target nucleic acid sequence in a cell. In certain embodiments, the method includes introducing into the cell a guide RNA comprising a portion that is complementary to all or a portion of the target nucleic acid sequence, introducing into the cell a Cas9 transposase fusion protein, and introducing into the cell a donor nucleic acid sequence, wherein the guide RNA and the Cas9 transposase fusion protein co-localize at the target nucleic acid sequence, wherein the Cas9 transposase fusion protein cleaves the target nucleic acid sequence and the donor nucleic acid sequence is inserted into the target nucleic acid sequence in a site specific manner. In some embodiments, the Cas9 transposase fusion protein comprises a portion of Cas9 protein, its variants or functional equivalents. In some embodiments, the Cas9 transposase fusion protein facilitates site specific integration of the donor nucleic acid sequence into the target nucleic acid sequence. In other embodiments, the guide RNA and Cas9 transposase fusion protein are each introduced to the cell via a vector comprising nucleic acid encoding the guide RNA and the Cas9 transposase fusion protein. In one embodiment, the Cas9 transposase fusion protein is introduced to the cell via a vector comprising nucleic acid encoding the fusion protein. In one embodiment, the vector is a plasmid. In some embodiments, a plurality of guide RNAs that are complementary to different target nucleic acid sequences are provided to the cell and wherein different target nucleic acid sequences are altered. In one embodiment, expression of the Cas9 transposase fusion protein is inducible. In some embodiments, the nucleic acid sequences encoding the guide RNA and/or the Cas9 transposase fusion protein are introduced to the cell via transfection or electroporation. In one embodiment, Cas9 is fused to a piggyBac transposase. In another embodiment, Cas9 is fused to a hyperactive piggyBac transposase. In one embodiment, the Cas9 portion of the Cas9 transposase fusion protein is nuclease competent. In one embodiment, the donor nucleic acid sequence is introduced into the cell by transfection or electroporation. In one embodiment, the donor nucleic acid sequence is introduced into the cell as a single stranded nucleic acid. In another embodiment, the donor nucleic acid sequence is introduced into the cell as a double stranded nucleic acid. In exemplary embodiment, the donor nucleic acid sequence is a transposon sequence. In one embodiment, the cell is from an embryo. In certain embodiments, the cell is a stem cell, zygote, or a germ line cell. In some embodiments, the stem cell is an embryonic stem cell or pluripotent stem cell. In one embodiment, the cell is a somatic cell. In another embodiment, the somatic cell is a eukaryotic cell. In one embodiment, the eukaryotic cell is an animal cell. In another embodiment, the animal cell is a porcine cell. In one embodiment, the porcine cell is a porcine fibroblast cell. In one embodiment, the guide RNA is about 10 to about 1000 nucleotides. In another embodiment, the guide RNA is about 15 to about 200 nucleotides.

According to another aspect, the present disclosure provides nucleic acid constructs. In one embodiment, the nucleic acid construct encodes a guide RNA comprising a portion that is complementary to all or a portion of a target nucleic acid sequence in a cell. In another embodiment, the nucleic acid construct encodes a Cas9 transposase fusion protein. In still another embodiment, the nucleic acid construct encodes a donor nucleic acid sequence for site specific integration into a target nucleic acid sequence in a cell. In an exemplary embodiment, the donor nucleic acid sequence is a transposon sequence. In one embodiment, transposon sequence is a piggyBac transposon sequence.

According to yet another aspect, the present disclosure provides an engineered cell. In one embodiment, the cell includes a guide RNA that comprise a portion that is complementary to all or a portion of a target nucleic acid sequences of the cell, a Cas9 transposase fusion protein, and a donor nucleic acid sequence, wherein the guide RNA and the Cas9 transposase fusion protein co-localize at the target nucleic acid sequence, wherein the Cas9 transposase fusion protein cleaves the target nucleic acid sequence and the donor nucleic acid sequence is inserted into the target nucleic acid sequence in a site specific manner In another embodiment, the donor nucleic acid sequence is a transposon sequence. In one embodiment, Cas9 is fused to a piggyBac transposase. In another embodiment, Cas9 is fused to a hyperactive piggyBac transposase. In one embodiment, the Cas9 portion of the Cas9 transposase fusion protein is nuclease competent. In another embodiment, the transposon sequence is a piggyBac transposon sequence.

According to one aspect, the RNA is between about 10 to about 1000 nucleotides. According to one aspect, the RNA is between about 20 to about 100 nucleotides.

According to one aspect, the one or more RNAs is a guide RNA. According to one aspect, the one or more RNAs is a tracrRNA-crRNA fusion.

According to one aspect, the DNA is genomic DNA, mitochondrial DNA, viral DNA, or exogenous DNA.

Further features and advantages of certain embodiments of the present invention will become more fully apparent in the following description of embodiments and drawings thereof, and from the claims.

### BRIEF DESCRIPTION OF THE DRAWINGS

The foregoing and other features and advantages of the present embodiments will be more fully understood from the following detailed description of illustrative embodiments taken in conjunction with the accompanying drawings in which:

FIG. 1 depicts a schematic diagram illustrating validating site-specific insertion of a 20 kb transposon sequence in porcine ROSA26 locus using junction PCR. Primer binding sites are indicated by small grey arrows.

FIGS. 2A and 2B depict the result of the integrated transposon-to-genome junction sequences captured by PCR. FIG. 2A discloses SEQ ID NOS 8-14, respectively, in order of appearance. FIG. 2B discloses SEQ ID NOS 15-22, respectively, in order of appearance.

### DETAILED DESCRIPTION

Aspects of the present disclosure relate to design, production, and use of fusion proteins involving a transposase and a sequence-specific nuclease to achieve efficient, site-specific integration of genetic elements of widely ranging sizes without the requirement for homology between the payload and the desired site of insertion. Embodiments of the present disclosure included engineered sequence-specific nucleases comprising sequence-specific DNA-binding domains fused to a non-specific DNA cleavage module. In some embodiments, the present disclosure includes zinc-finger nucleases (ZFNs), which are fusions of the non-specific DNA cleavage domain from the FokI restriction endonuclease with zinc-finger proteins. ZFN dimers induce targeted DNA double-stranded breaks (DSBs) that stimulate DNA damage response pathway. The binding specificity of the designed zinc-finger domain directs the ZFN to a genomic site. In other embodiments, the present disclosure includes transcription activator-like effector (TALE) nucleases (TALENs), which are fusions of the FokI cleavage domain and DNA-binding domains derived from TALE proteins. TALEs contain multiple 33-35 amino acid repeat domains that each recognizes a single base pair. Like ZFNs, TALENs induce targeted DSBs that activate DNA damage response pathways and enable custom alterations of the target genomic loci. In exemplary embodiments, the present disclosure includes clustered regulatory interspaced short palindromic repeats (CRISPR)/Cas associated systems as sequence-specific nucleases. CRISPR are loci that contain multiple short direct repeats that are known to provide acquired immunity to bacteria and archaea. CRISPR systems rely on crRNA and tracrRNA for sequence-specific silencing of invading foreign DNA. Three types of CRISPR/Cas systems exist: In type II systems, Cas9 serves as an RNA-guided DNA endonuclease that cleaves DAN upon crRNA-tracrRNA target recognition. According to certain exemplary embodiments, these sequence-specific nucleases are used to generate fusion proteins with transposases. These fusion proteins are expressed in cells and produce site-specific double-stranded breaks in a host genome or target DNA. The DSBs can be repaired by non-homologous end joining (NHEJ) or homology directed repair (HDR) mechanisms. The transposase of the fusion protein is responsible for integrating foreign or donor nucleic acid sequence at the target site by NHEJ which ligates or joins two broken ends together. NHEJ does not use a homologous template for repair and typically leads to the introduction of small insertions and deletions at the site of the break.

Aspects of the present invention are directed to the use of CRISPR/Cas9 and transposase fusion protein for genome engineering. Specifically, the clustered regularly interspaced short palindromic repeats (CRISPR) and CRISPR associated genes (Cas genes), referred to herein as the CRISPR/Cas system, and in combination with transposases, has been adapted as an efficient gene targeting and genome engineering technology.

A comparison of the predominant methods of genetic insertion are compared in the table below.

| | Insertion | | Size | Time |
|---|---|---|---|---|
| System | site | Efficiency | constraint | scale |
| Lentivirus | random | high | <10 kb | weeks |
| Transposon/ | random | high | up to 200 | days to |
| transposase | | | kb | weeks |
| Homologous | site-specific | very | several | months |
| recombination | | low | kilobases | |
| Homology directed | site-specific | low | several | weeks to |
| repair | | | kilobases | months |
| Recombinase | site-specific | medium | tens of | months |
| | | | kilobases | |
| The present | site-specific | high | up to 200 | days to |
| disclosure | | | kb | weeks |

### Cas9 Description

RNA guided DNA binding proteins are readily known to those of skill in the art to bind to DNA for various purposes. Such DNA binding proteins may be naturally occurring. DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety.

In general, bacterial and archaeal CRISPR-Cas systems rely on short guide RNAs in complex with Cas proteins to direct degradation of complementary sequences present within invading foreign nucleic acid. See Deltcheva, E. et al. CRISPR RNA maturation by trans-encoded small RNA and host factor RNase III. Nature 471, 602-607 (2011); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., Phage response to CRISPR-encoded resistance in Streptococcus thermophilus. Journal of Bacteriology 190, 1390 (February 2008).

Three classes of CRISPR systems are generally known and are referred to as Type I, Type II or Type III). According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (June 2011) hereby incorporated by reference in its entirety. Within bacteria, the Type II effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9. TracrRNA-crRNA fusions are contemplated for use in the present methods.

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see P. Horvath, R. Barrangou, CRISPR/Cas, the immune system of bacteria and archaea. Science 327, 167 (Jan. 8, 2010) hereby incorporated by reference in its entirety and NNAGAAW (see H. Deveau et al., Phage response to CRISPR-encoded resistance in Streptococcus thermophilus. Journal of bacteriology 190, 1390 (February 2008) hereby incorporated by reference in its entirety), respectively, while different S. mutans systems tolerate NGG or NAAR (see J. R. van der Ploeg, Analysis of CRISPR in Streptococcus mutans suggests frequent occurrence of acquired immunity against infection by M102-like bacteriophages. Microbiology 155, 1966 (June 2009) hereby incorporated by reference in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al., Analysis of streptococcal CRISPRs from human saliva reveals substantial sequence diversity within and between subjects over time. Genome research 21, 126 (January 2011) each of which are hereby incorporated by reference in their entireties.

In S. pyogenes, Cas9 generates a blunt-ended double-stranded break 3 bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinek et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi1; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pa1 5 FAPERJ; Gluconacetobacter diazotrophicus Pa1 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha 14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csn1. An exemplary S. pyogenes Cas9 protein sequence is provided in Deltcheva et al., Nature 471, 602-607 (2011) hereby incorporated by reference in its entirety.

Modification to the Cas9 protein is contemplated by the present disclosure. CRISPR systems useful in the present disclosure are described in R. Barrangou, P. Horvath, CRISPR: new horizons in phage resistance and strain identification. Annual review of food science and technology 3, 143 (2012) and B. Wiedenheft, S. H. Sternberg, J. A. Doudna, RNA-guided genetic silencing systems in bacteria and archaea. Nature 482, 331 (Feb. 16, 2012) each of which are hereby incorporated by reference in their entireties.

According to certain aspects, the DNA binding protein is altered or otherwise modified to inactivate the nuclease activity. Such alteration or modification includes altering one or more amino acids to inactivate the nuclease activity or the nuclease domain. Such modification includes removing the polypeptide sequence or polypeptide sequences exhibiting nuclease activity, i.e. the nuclease domain, such that the polypeptide sequence or polypeptide sequences exhibiting nuclease activity, i.e. nuclease domain, are absent from the DNA binding protein. Other modifications to inactivate nuclease activity will be readily apparent to one of skill in the art based on the present disclosure. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. Accordingly, the DNA binding protein includes the polypeptide sequence or sequences required for DNA binding but may have one or more or all of the nuclease sequences exhibiting nuclease activity inactivated.

According to one aspect, a DNA binding protein having two or more nuclease domains may be modified or altered to inactivate all but one of the nuclease domains. Such a modified or altered DNA binding protein is referred to as a DNA binding protein nickase, to the extent that the DNA binding protein cuts or nicks only one strand of double stranded DNA. When guided by RNA to DNA, the DNA binding protein nickase is referred to as an RNA guided DNA binding protein nickase. An exemplary DNA binding protein is an RNA guided DNA binding protein nuclease of a Type II CRISPR System, such as a Cas9 protein or modified Cas9 or homolog of Cas9. An exemplary DNA binding protein is a Cas9 protein nickase. An exemplary DNA binding protein is an RNA guided DNA binding protein of a Type II CRISPR System which lacks nuclease activity. An exemplary DNA binding protein is a nuclease-null or nuclease deficient Cas9 protein.

According to an additional aspect, nuclease-null Cas9 proteins are provided where one or more amino acids in Cas9 are altered or otherwise removed to provide nuclease-null Cas9 proteins. According to one aspect, the amino acids include D10 and H840. See Jinek et al., Science 337, 816-821 (2012). According to an additional aspect, the amino acids include D839 and N863. According to one aspect, one or more or all of D10, H840, D839 and H863 are substituted with an amino acid which reduces, substantially eliminates or eliminates nuclease activity. According to one aspect, one or more or all of D10, H840, D839 and H863 are substituted with alanine. According to one aspect, a Cas9 protein having one or more or all of D10, H840, D839 and H863 substituted with an amino acid which reduces, substantially eliminates or eliminates nuclease activity, such as alanine, is referred to as a nuclease-null Cas9 (“Cas9Nuc”) and exhibits reduced or eliminated nuclease activity, or nuclease activity is absent or substantially absent within levels of detection. According to this aspect, nuclease activity for a Cas9Nuc may be undetectable using known assays, i.e. below the level of detection of known assays.

According to one aspect, the Cas9 protein, Cas9 protein nickase or nuclease null Cas9 includes homologs and orthologs thereof which retain the ability of the protein to bind to the DNA and be guided by the RNA. According to one aspect, the Cas9 protein includes the sequence as set forth for naturally occurring Cas9 from S. thermophiles or S. pyogenes and protein sequences having at least 30%, 40%, 50%, 60%, 70%, 80%, 90%, 95%, 98% or 99% homology thereto and being a DNA binding protein, such as an RNA guided DNA binding protein.

An exemplary CRISPR system includes the S. thermophiles Cas9 nuclease (ST1 Cas9) (see Esvelt K M, et al., Orthogonal Cas9 proteins for RNA-guided gene regulation and editing, Nature Methods., (2013) hereby incorporated by reference in its entirety). An exemplary CRISPR system includes the S. pyogenes Cas9 nuclease (Sp. Cas9), an extremely high-affinity (see Sternberg, S. H., Redding, S., Jinek, M., Greene, E. C. & Doudna, J. A. DNA interrogation by the CRISPR RNA-guided endonuclease Cas9. Nature 507, 62-67 (2014) hereby incorporated by reference in its entirety), programmable DNA-binding protein isolated from a type II CRISPR-associated system (see Garneau, J. E. et al. The CRISPR/Cas bacterial immune system cleaves bacteriophage and plasmid DNA. Nature 468, 67-71 (2010) and Jinek, M. et al. A programmable dual-RNA-guided DNA endonuclease in adaptive bacterial immunity. Science 337, 816-821 (2012) each of which are hereby incorporated by reference in its entirety). According to certain aspects, a nuclease null or nuclease deficient Cas 9 can be used in the methods described herein. Such nuclease null or nuclease deficient Cas9 proteins are described in Gilbert, L. A. et al. CRISPR-mediated modular RNA-guided regulation of transcription in eukaryotes. Cell 154, 442-451 (2013); Mali, P. et al. CAS9 transcriptional activators for target specificity screening and paired nickases for cooperative genome engineering. Nature biotechnology 31, 833-838 (2013); Maeder, M.L. et al. CRISPR RNA-guided activation of endogenous human genes. Nature methods 10, 977-979 (2013); and Perez-Pinera, P. et al. RNA-guided gene activation by CRISPR-Cas9-based transcription factors. Nature methods 10, 973-976 (2013) each of which are hereby incorporated by reference in its entirety. The DNA locus targeted by Cas9 (and by its nuclease-deficient mutant, “dCas9” precedes a three nucleotide (nt) 5′-NGG-3′ “PAM” sequence, and matches a 15-22-nt guide or spacer sequence within a Cas9-bound RNA cofactor, referred to herein and in the art as a guide RNA. Altering this guide RNA is sufficient to target Cas9 or a nuclease deficient Cas9 to a target nucleic acid. In a multitude of CRISPR-based biotechnology applications (see Mali, P., Esvelt, K. M. & Church, G. M. Cas9 as a versatile tool for engineering biology. Nature methods 10, 957-963 (2013); Hsu, P. D., Lander, E. S. & Zhang, F. Development and Applications of CRISPR-Cas9 for Genome Engineering. Cell 157, 1262-1278 (2014); Chen, B. et al. Dynamic imaging of genomic loci in living human cells by an optimized CRISPR/Cas system. Cell 155, 1479-1491 (2013); Shalem, O. et al. Genome-scale CRISPR-Cas9 knockout screening in human cells. Science 343, 84-87 (2014); Wang, T., Wei, J. J., Sabatini, D. M. & Lander, E. S. Genetic screens in human cells using the CRISPR-Cas9 system. Science 343, 80-84 (2014); Nissim, L., Perli, S. D., Fridkin, A., Perez-Pinera, P. & Lu, T. K. Multiplexed and Programmable Regulation of Gene Networks with an Integrated RNA and CRISPR/Cas Toolkit in Human Cells. Molecular cell 54, 698-710 (2014); Ryan, O. W. et al. Selection of chromosomal DNA libraries using a multiplex CRISPR system. eLife 3 (2014); Gilbert, L. A. et al. Genome-Scale CRISPR-Mediated Control of Gene Repression and Activation. Cell (2014); and Citorik, R. J., Mimee, M. & Lu, T. K. Sequence-specific antimicrobials using efficiently delivered RNA-guided nucleases. Nature biotechnology (2014) each of which are hereby incorporated by reference in its entirety), the guide is often presented in a so-called sgRNA (single guide RNA), wherein the two natural Cas9 RNA cofactors (gRNA and tracrRNA) are fused via an engineered loop or linker.

According to one aspect, the Cas9 protein is an enzymatically active Cas9 protein, a Cas9 protein wild-type protein, a Cas9 protein nickase or a nuclease null or nuclease deficient Cas9 protein. Additional exemplary Cas9 proteins include Cas9 proteins attached to, bound to or fused with functional proteins such as transcriptional regulators, such as transcriptional activators or repressors, a Fok-domain, such as Fok 1, an aptamer, a binding protein, PP7, MS2 and the like.

According to certain aspects, the Cas9 protein may be delivered directly to a cell by methods known to those of skill in the art, including injection or lipofection, or as translated from its cognate mRNA, or transcribed from its cognate DNA into mRNA (and thereafter translated into protein). Cas9 DNA and mRNA may be themselves introduced into cells through electroporation, transient and stable transfection (including lipofection) and viral transduction or other methods known to those of skill in the art.

### Guide RNA Description

Embodiments of the present disclosure are directed to the use of a CRISPR/Cas system and, in particular, a guide RNA which may include one or more of a spacer sequence, a tracr mate sequence and a tracr sequence. The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed an sgRNA).

According to certain aspects, the guide RNA is between about 10 to about 500 nucleotides. According to one aspect, the guide RNA is between about 20 to about 100 nucleotides. According to certain aspects, the spacer sequence is between about 10 and about 500 nucleotides in length. According to certain aspects, the tracr mate sequence is between about 10 and about 500 nucleotides in length. According to certain aspects, the tracr sequence is between about 10 and about 100 nucleotides in length. According to certain aspects, the linker nucleic acid sequence is between about 10 and about 100 nucleotides in length.

According to one aspect, embodiments described herein include guide RNA having a length including the sum of the lengths of a spacer sequence, tracr mate sequence, tracr sequence, and linker sequence (if present). Accordingly, such a guide RNA may be described by its total length which is a sum of its spacer sequence, tracr mate sequence, tracr sequence, and linker sequence (if present). According to this aspect, all of the ranges for the spacer sequence, tracr mate sequence, tracr sequence, and linker sequence (if present) are incorporated herein by reference and need not be repeated. A guide RNA as described herein may have a total length based on summing values provided by the ranges described herein. Aspects of the present disclosure are directed to methods of making such guide RNAs as described herein by expressing constructs encoding such guide RNA using promoters and terminators and optionally other genetic elements as described herein.

According to certain aspects, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

### Donor Description

The term “donor nucleic acid” include a nucleic acid sequence which is to be inserted into genomic DNA according to methods described herein. The donor nucleic acid sequence may be expressed by the cell.

According to one aspect, the donor nucleic acid is exogenous to the cell. According to one aspect, the donor nucleic acid is foreign to the cell. According to one aspect, the donor nucleic acid is non-naturally occurring within the cell. According to one aspect, the donor nucleic acid is a transposon sequence.

### Transcription Regulator Description

According to one aspect, an engineered Cas9-gRNA system is provided which enables RNA-guided DNA regulation in cells by tethering transcriptional activation/repression domains to either a nuclease-null Cas9 or to guide RNAs. According to one aspect of the present disclosure, one or more transcriptional regulatory proteins or domains (such terms are used interchangeably) are joined or otherwise connected to a nuclease-deficient Cas9 or one or more guide RNA (gRNA). The transcriptional regulatory domains correspond to targeted loci. Accordingly, aspects of the present disclosure include methods and materials for localizing transcriptional regulatory domains to targeted loci by fusing, connecting or joining such domains to either Cas9N or to the gRNA.

### Foreign Nucleic Acids Description

Foreign nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

### Cells

Cells according to the present disclosure include any cell into which foreign nucleic acids can be introduced and expressed as described herein. It is to be understood that the basic concepts of the present disclosure described herein are not limited by cell type. In some embodiments, the cell is from an embryo. The cell can be a stem cell, zygote, or a germ line cell. In embodiments where the cell is a stem cell, the stem cell is an embryonic stem cell or pluripotent stem cell. In other embodiments, the cell is a somatic cell. In embodiments, where the cell is a somatic cell, the somatic cell is a eukaryotic cell or prokaryotic cell. The eukaryotic cell can be an animal cell, such as from a pig, mouse, rat, rabbit, dog, horse, cow, non-human primate, human In some embodiments, the animal cell is a porcine cell. In an exemplary embodiment, the porcine cell is a porcine fibroblast cell.

### Vectors

Vectors are contemplated for use with the methods and constructs described herein. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, doublestranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Methods of non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species.

### Regulatory Elements and Terminators and Tags

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EFla promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

The following examples are set forth as being representative of the present disclosure. These examples are not to be construed as limiting the scope of the present disclosure as these and other equivalent embodiments will be apparent in view of the present disclosure, figures and accompanying claims.

### EXAMPLES

### Example I

### CRISPR Cas9-Transposase Fusion Mediated Site Specific Integration of a 20 kb Transposon Sequence

To construct the fusion between the transposase and the sequence-specific nuclease, a hyperactive piggyBac transposase sequence (See, e.g., Yusa, K., Zhou, L., Li, M. A., Bradley, A. & Craig, N. L. A hyperactive piggyBac transposase for mammalian applications. Proc Natl Acad Sci U S A 108, 1531-1536 (2011), hereby incorporated by reference in its entirety) downstream of Cas9 was cloned into a pcDNA3.3 backbone vector using Gateway recombination (See, e.g., Chavez, A. et al. Highly efficient Cas9-mediated transcriptional programming Nat Meth 12, 326-328 (2015), hereby incorporated by reference in its entirety). Critically, the fusion construct involved the nuclease-competent version of Cas9, rather than the nuclease-null dCas9. The latter version has been used previously in many applications for which the function of Cas9 to localize to specific genetic sequences when in complex with guide RNAs (gRNAs) is desired but the nuclease function is not. In contrast, this example makes use of the nuclease-competent version of Cas9, which retains its capacities for both sequence-specific localization and cleavage of double-stranded DNA (dsDNA). In certain embodiments, linkers are included between Cas9 and the hyperactive piggyBac transposase. For example, the SV40 nuclear localization sequence and gateway attachment site downstream of Cas9 function as linkers. Additionally, there is a 6-amino acid linker (GSGSGS (glycine-serine-glycine-serine-glycine-serine) (SEQ ID NO: 1)) downstream of the gateway attachment site and upstream of the hyperactive piggyBac transposase.

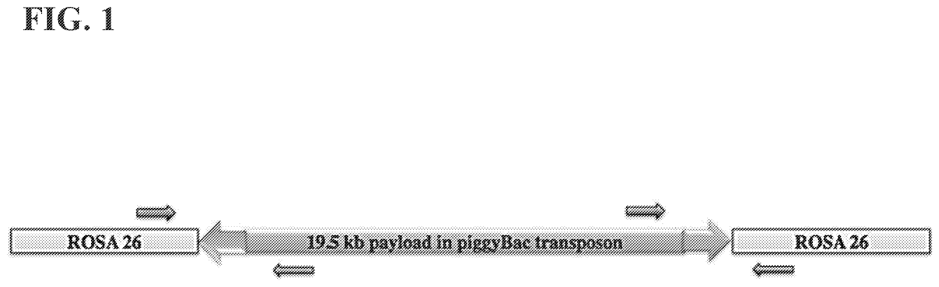

To test whether this construct could mediate site-specific integration of a piggyBac transposon containing a 20 kb payload, porcine fibroblast cells were nucleofected with a Cas9-piggyBac transposase fusion construct, a 20 kb piggyBac transposon (harboring a GFP reporter sequence), and a gRNA targeting the ROSA26 locus of the porcine genome. As a negative control, porcine fibroblast cells were nucleofected with a piggyBac transposase, the 20 kb piggyBac transposon, and a gRNA targeting the ROSA26 locus. The ROSA26 locus in the porcine genome is a “safe harbor” for transgene integration and expression, as it is ubiquitously transcribed independent of cell type. Five days after transfection, fluorescent cells were observed, indicating that in some proportion of the cells, the transposon had been integrated into the genome. Puromycin was applied to the culture medium for five additional days to select for cells containing the transposon. The cells were then collected, genomic DNA was extracted, and a junction PCR was performed to validate that the transposon had been inserted near the site specified by the gRNA (FIG. 1).

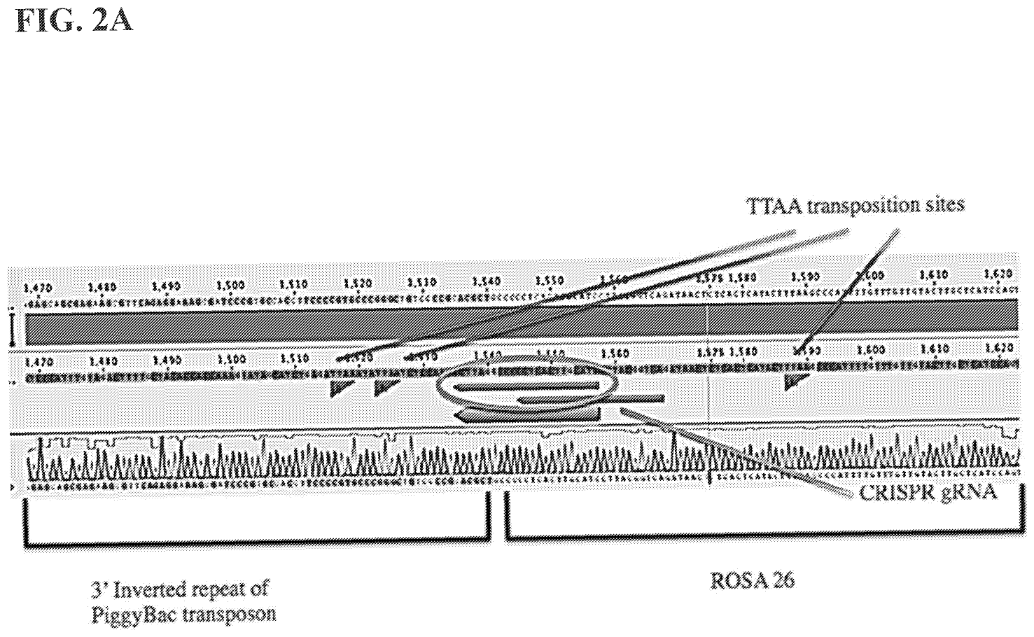

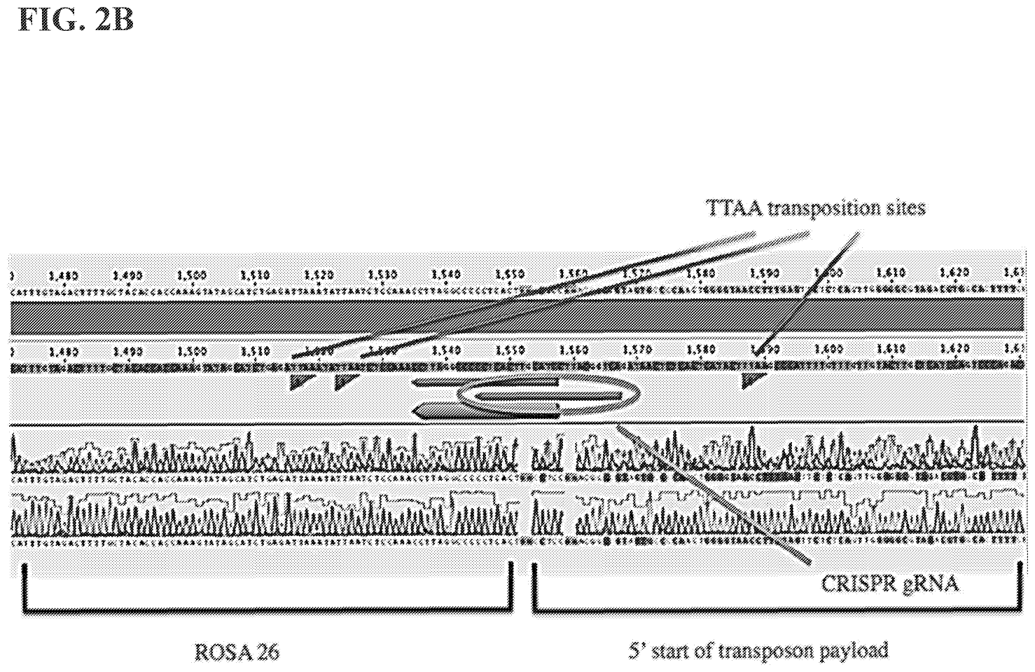

After sequencing the PCR products by Sanger sequencing, both 5′ and 3′ genomic junctions of the integrated transposon were identified (FIG. 2A and FIG. 2B). Interestingly, the transposon was found to be integrated at the site specified by the CRISPR gRNA, rather than at canonical TTAA piggyBac transposition sites. Furthermore, junctions did not contain the full-length inverted repeats of the piggyBac transposon. Instead, the inverted repeats were truncated, indicating a mechanism of integration dissimilar to canonical piggyBac transposition. The transposon payload, however, appeared intact. It was hypothesized that the mechanism of integration involves simultaneous cleavage of the ROSA26 locus by Cas9 and the transposon by the piggyBac transposase, followed by non-homologous end joining of the transposon at the site of Cas9 cleavage.

Embodiments of the present disclosure provide several transposase/transposon systems that can be used with CRISPR Cas system to direct site specific integration of large transposon sequence or elements into the host genome or target DNA. Non-limiting examples of the transposase/transposon systems include the piggyBac system, the Sleeping Beauty system, and the Tn5 system.

Embodiments of the present disclosure further provide sequence-specific nucleases including but not limited to CRISPR Cas9, variants of Cas9 or nucleases similar to Cas9 in function.

The present disclosure provides the identification and use of a novel mechanism for the integration of DNA elements that resembles neither canonical transposition nor homology-directed repair.

### Example II

### Methods

A list of gRNAs and primers used in this study:

ROSA gRNA 1:
(SEQ ID NO: 2)
5′-TGACCGTAAGGATGCAAGTG-3′
ROSA gRNA 2:
(SEQ ID NO: 3)
5′-GATGCAAGTGAGGGGGCCTA-3′
ROSA fw:
(SEQ ID NO: 4)
5′-CAG GCA ACA CCT AAG CCT GA-3′
ROSA rv:
(SEQ ID NO: 5)
5′-TTG GGC CTA TGC TCA AGA TG-3′
pb transposon fw:
(SEQ ID NO: 6)
5′-GCG ACA CGG AAA TGT TGA AT-3′
pb transposon rv:
(SEQ ID NO: 7)
5′-GCA ACC TCC CCT TCT ACG AG-3′

### Cell Culture

Porcine fibroblast cells were maintained in Dulbecco's modified Eagle's medium (DMEM, Invitrogen) high glucose with sodium pyruvate supplemented with 15% fetal bovine serum (Invitrogen), 1% HEPES, and 1% penicillin/streptomycin (Pen/Strep, Invitrogen). All cells were maintained in a humidified incubator at 37° C. and 5% CO₂.

Nucleofection 30 μg total DNA in equimolar ratios was delivered to porcine fibroblast cells using a 4D nucleofector (Lonza, 4D nucleofector). Briefly, one million cells were mixed with 82 μL P3 solution and 18 μL supplement, transferred to a cuvette, and shocked twice using pulse code CA137. Transfected cells were resuspended in warm media using a transfer pipette and seeded in cell culture flasks.

### Junction PCR and Sequencing

25 μl PCR reactions contained 12.5 μl 2× KAPA Hifi Hotstart ReadyMix (KAPA Biosystems), 100 nM primers, and 7.5 μl water. Reactions were incubated at 95° C. for 5 min followed by 32 cycles of 98° C., 20 s; 60° C., 20 s and 72° C., 50 s. PCR products were checked on EX 2% gels (Invitrogen), and bright bands were purified (QlAquick Gel Extraction Kit), TOPO cloned (Invitrogen), and sequenced by Sanger sequencing (Genewiz LLC).

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of altering a target nucleic acid sequence in a cell comprising:
introducing into the cell a guide RNA comprising a portion that is complementary to all or a portion of the target nucleic acid sequence,
introducing into the cell a Cas9 transposase fusion protein, and
introducing into the cell a donor nucleic acid sequence,
wherein the guide RNA and the Cas9 transposase fusion protein co-localize at the target nucleic acid sequence, wherein the Cas9 transposase fusion protein cleaves the target nucleic acid sequence and the donor nucleic acid sequence is inserted into the target nucleic acid sequence in a site specific manner.

2. The method of claim 1 wherein the Cas9 transposase fusion protein comprises a portion of Cas9 protein, its variants or functional equivalents.

3. The method of claim 1 wherein the Cas9 transposase fusion protein facilitates site specific integration of the donor nucleic acid sequence into the target nucleic acid sequence.

4. The method of claim 1 wherein the guide RNA and Cas9 transposase fusion protein are each introduced to the cell via a vector comprising nucleic acid encoding the guide RNA and the Cas9 transposase fusion protein.

5. The method of claim 4 wherein the vector is a plasmid.

6. The method of claim 1 wherein the Cas9 transposase fusion protein is introduced to the cell via a vector comprising nucleic acid encoding the fusion protein.

7. The method of claim 6 wherein the vector is a plasmid.

8. The method of claim 1 wherein a plurality of guide RNAs that are complementary to different target nucleic acid sequences are provided to the cell and wherein different target nucleic acid sequences are altered.

9. The method of claim 1 wherein expression of the Cas9 transposase fusion protein is inducible.

10. The method of claim 1 wherein the introducing step comprising transfecting or electroporating nucleic acid sequences encoding the guide RNA and/or the Cas9 transposase fusion protein.

11. The method of claim 1 wherein the donor nucleic acid sequence is introduced into the cell by transfection or electroporation.

12. The method of claim 1 wherein the donor nucleic acid sequence is introduced into the cell as a single stranded nucleic acid.

13. The method of claim 1 wherein the donor nucleic acid sequence is introduced into the cell as a double stranded nucleic acid.

14. The method of claim 1 wherein the donor nucleic acid sequence is a transposon sequence.

15. The method of claim 14 wherein the donor nucleic acid sequence is a transposon sequence.

16. The method of claim 1 wherein the cell is from an embryo.

17. The method of claim 1 wherein the cell is a stem cell, zygote, or a germ line cell.

18. The method of claim 17 wherein the stem cell is an embryonic stem cell or pluripotent stem cell.

19. The method of claim 1 wherein the cell is a somatic cell.

20. The method of claim 19 wherein the somatic cell is a eukaryotic cell.

21. The method of claim 20 wherein the eukaryotic cell is an animal cell.

22. The method of claim 21 wherein the animal cell is a porcine cell.

23. The method of claim 22 wherein the porcine cell is a porcine fibroblast cell.

24. The method of claim 1 wherein the guide RNA is about 10 to about 1000 nucleotides.

25. The method of claim 1 wherein the guide RNA is about 15 to about 200 nucleotides.

26. A nucleic acid construct encoding a guide RNA comprising a portion that is complementary to all or a portion of a target nucleic acid sequence in a cell.

27. A nucleic acid construct encoding a Cas9 transposase fusion protein.

28. A nucleic acid construct encoding a donor nucleic acid sequence for site specific integration into a target nucleic acid sequence in a cell.

29. The nucleic acid construct of claim 28 wherein the donor nucleic acid sequence is a transposon sequence.

30. The nucleic acid construct of claim 29, wherein the transposon sequence is a piggyBac transposon sequence.

31. The method of claim 1, wherein Cas9 is fused to a piggyBac transposase.

32. The method of claim 31, wherein Cas9 is fused to a hyperactive piggyBac transposase.

33. The method of claim 1, wherein the Cas9 portion of the Cas9 transposase fusion protein is nuclease competent.

34. An engineered cell comprising:
a guide RNA that comprise a portion that is complementary to all or a portion of a target nucleic acid sequences of the cell,
a Cas9 transposase fusion protein, and
a donor nucleic acid sequence,
wherein the guide RNA and the Cas9 transposase fusion protein co-localize at the target nucleic acid sequence, wherein the Cas9 transposase fusion protein cleaves the target nucleic acid sequence and the donor nucleic acid sequence is inserted into the target nucleic acid sequence in a site specific manner.

35. The engineered cell of claim 34 wherein the donor nucleic acid sequence is a transposon sequence.

36. The engineered cell of claim 34, wherein Cas9 is fused to a piggyBac transposase.

37. The engineered cell of claim 36, wherein Cas9 is fused to a hyperactive piggyBac transposase.

38. The engineered cell of claim 34, wherein the Cas9 portion of the Cas9 transposase fusion protein is nuclease competent.

39. The engineered cell of claim 37, wherein the transposon sequence is a piggyBac transposon sequence.


SEQUENCE LISTING (22 sequences)

SEQ ID NO: 1 - protein, 6 aa, Artificial Sequence
  Description of Artificial Sequence Synthetic peptide
GSGSGS

SEQ ID NO: 2 - DNA, 20 bp, Artificial Sequence
  Description of Artificial Sequence Synthetic primer
tgaccgtaaggatgcaagtg

SEQ ID NO: 3 - DNA, 20 bp, Artificial Sequence
  Description of Artificial Sequence Synthetic primer
gatgcaagtgagggggccta

SEQ ID NO: 4 - DNA, 20 bp, Artificial Sequence
  Description of Artificial Sequence Synthetic primer
caggcaacacctaagcctga

SEQ ID NO: 5 - DNA, 20 bp, Artificial Sequence
  Description of Artificial Sequence Synthetic primer
ttgggcctatgctcaagatg

SEQ ID NO: 6 - DNA, 20 bp, Artificial Sequence
  Description of Artificial Sequence Synthetic primer
gcgacacggaaatgttgaat

SEQ ID NO: 7 - DNA, 20 bp, Artificial Sequence
  Description of Artificial Sequence Synthetic primer
gcaacctccccttctacgag

SEQ ID NO: 8 - DNA, 81 bp, Artificial Sequence
  Description of Artificial Sequence Synthetic oligonucleotide
gagcagcgagaagcgttcagaggaaagcgatcccgtgccaccttccccgtgcccgggctgtccccgcacgctgccccctca

SEQ ID NO: 9 - DNA, 60 bp, Artificial Sequence
  Description of Artificial Sequence Synthetic oligonucleotide
gtcagataactctcactcatactttaagcccattttgtttgttgtacttgctcatccagt

SEQ ID NO: 10 - DNA, 64 bp, Artificial Sequence
  Description of Artificial Sequence Synthetic oligonucleotide
tcccatttgtagacttttgctacaccaccaaagtatagcatctgagattaaatattaatctcca

SEQ ID NO: 11 - DNA, 21 bp, Artificial Sequence
  Description of Artificial Sequence Synthetic oligonucleotide
cttaggccccctcacttgcat

SEQ ID NO: 12 - DNA, 64 bp, Artificial Sequence
  Description of Artificial Sequence Synthetic oligonucleotide
tacggtcagataactctcactcatactttaagcccattttgtttgttgtacttgctcatccagt

SEQ ID NO: 13 - DNA, 119 bp, Artificial Sequence
  Description of Artificial Sequence Synthetic polynucleotide
gagcagcgagaagcgttcagaggaaagcgatcccgtgccaccttccccgtgcccgggctgtccccgcacgctgccccctcacttgcatccttacggtcagataactctcactcatactt

SEQ ID NO: 14 - DNA, 33 bp, Artificial Sequence
  Description of Artificial Sequence Synthetic oligonucleotide
gcccattttgtttgttgtacttgctcatccagt

SEQ ID NO: 15 - DNA, 82 bp, Artificial Sequence
  Description of Artificial Sequence Synthetic oligonucleotide
  modified_base (81)..(82): a, c, t, g, unknown or other
catttgtagacttttgctacaccaccaaagtatagcatctgagattaaatattaatctccaaaccttaggccccctcactnn

SEQ ID NO: 16 - DNA, 30 bp, Artificial Sequence
  Description of Artificial Sequence Synthetic oligonucleotide
gtagtgccccaactggggtaacctttgagt

SEQ ID NO: 17 - DNA, 33 bp, Artificial Sequence
  Description of Artificial Sequence Synthetic oligonucleotide
ctctcagttgggggcgtagacgtggcacttttc

SEQ ID NO: 18 - DNA, 159 bp, Artificial Sequence
  Description of Artificial Sequence Synthetic polynucleotide
catttgtagacttttgctacaccaccaaagtatagcatctgagattaaatattaatctccaaaccttaggccccctcacttgcatccttacggtcagataactctcactcatactttaagcccattttgtttgttgtacttgctcatccagtcccagac

SEQ ID NO: 19 - DNA, 106 bp, Artificial Sequence
  Description of Artificial Sequence Synthetic polynucleotide
  modified_base (81)..(82): a, c, t, g, unknown or other
  modified_base (88)..(89): a, c, t, g, unknown or other
catttgtagacttttgctacaccaccaaagtatagcatctgagattaaatattaatctccaaaccttaggccccctcactnncctccnnacggccgtagtgcccca

SEQ ID NO: 20 - DNA, 51 bp, Artificial Sequence
  Description of Artificial Sequence Synthetic oligonucleotide
tggggtaacctttgagttctctcagttgggggcgtagacgtggcacttttc

SEQ ID NO: 21 - DNA, 120 bp, Artificial Sequence
  Description of Artificial Sequence Synthetic polynucleotide
  modified_base (81)..(82): a, c, t, g, unknown or other
  modified_base (88)..(89): a, c, t, g, unknown or other
catttgtagacttttgctacaccaccaaagtatagcatctgagattaaatattaatctccaaaccttaggccccctcactnncctccnnacggccgtagtgccccaactggggtaacctt

SEQ ID NO: 22 - DNA, 37 bp, Artificial Sequence
  Description of Artificial Sequence Synthetic oligonucleotide
agttctctcagttgggggcgtagacgtggcacttttc